(19) United States
(12) Patent Application Publication
(10) Pub. No.: US 2024/0122455 A1
(43) Pub. Date: Apr. 18, 2024

(54) AIR RETENTION DEVICE AND PATCH FOR COLONOSCOPY

(71) Applicant: ANDREW S. KORMAN, LIVINGSTON, NJ (US)

(72) Inventor: ANDREW S. KORMAN, LIVINGSTON, NJ (US)

(21) Appl. No.: 17/967,777

(22) Filed: Oct. 17, 2022

(51) Int. Cl. A61B 1/00; A61B 1/31

(52) U.S. Cl. CPC A61B 1/00131; A61B 1/31

(57) ABSTRACT

An improved air retention device or a patch for colonoscopy comprises of a first end and a second end located at a distal end of the first end. During colonoscopy, the first end of the air retention device is put on the surface of a person's buttocks such that the surface of the first end is flush with the skin of the person's buttocks—the device placed on the gluteus of a person and tightened around a colonoscope. The second end of the air retention device is pulled away from the first end around a central axis aligned with the center of the anus of the person undergoing the colonoscopy.

### FIELD OF THE DISCLOSURE

The present disclosure generally relates to an air retention device and patch for colonoscopy and method of using the same. More specifically, the present disclosure is directed to an improved air retention device and patch shaped and configured to reduce water and air/carbon dioxide during the colonoscopy procedure.

### BACKGROUND

Colonoscopy is a procedure designed to examine the rectum and large intestine (colon) for both diagnostic and therapeutic purposes. The field of the present invention relates generally to an improved device and method of using the device during the colonoscopic examination of a human colon. A long, flexible tube about the width of a finger with a light and small video camera on the end is inserted through the anus and into the rectum and colon of a person during the colonoscopic examination, as further discussed below.

Colonoscopy is generally a very sensitive and specific means for examining the colon, particularly for the diagnosis of colon cancers and polyps. The structure of the colon, specifically the cecum—the portion of the colon farthest from the anus—can be a common location for cancer; it is generally desirable that the entire colon be completely examined. During a typical colonoscopy procedure, a device (generally a colonoscope or an endoscope) is brought in proximity and inserted into the anus of a patient, through the rectum, and then advanced through the sigmoid colon, descending colon, transverse colon, ascending colon and into the cecum. Examination using an endoscope or colonoscope is widely used for observing, photographing a part of the examination, taking specimens from a lesion to ascertain if a tumor is malignant, removing when the tumor is malignant, observing other pathological and physiological conditions and processes, and removing foreign body. An endoscope or colonoscope is also useful in the examination of a colon.

In the conventional method of examining a colon using an endoscope, the endoscope must be manually inserted into the colon by pushing it with the hands, even though various other techniques can be applied.

Various prior art devices and methods are directed to perform a colonoscopy by manually directing and advancing a flexible tube (the colonoscope) through the colon. Conventional and prior art colonoscopy-related devices have multiple disadvantages.

One key disadvantage in prior art is that the technique used is cumbersome and requires significant dexterity and training for medical staff to perform the procedure. In some current designs and configurations, the scope often partially returns to a neutral position when the directional control dials are released during the colonoscopy procedure. Control dials in current scopes usually employ control wires which are attached to the tip of the scope. These wires bend the tip of the scope when they are tightened. Advancing the scope, which is typically about 160 centimeters in length, can be difficult due to a loop or bend in the sigmoid colon.

During the colonoscopy procedure, the advancement of the scope often results in the formation of a loop along the shaft of the flexible scope. This looping occurs most commonly in the sigmoid colon and, though usually much less frequently, in the transverse colon. Failure to substantially straighten the loop in the sigmoid colon prior to continuing to advance the scope can cause enlargement of the loop and result in even more difficulty in advancing the scope. This enlarged looping can result in patient pain and damage, including cardiovascular reactions such as hypotension and bradycardia. Forceful pushing of the scope in the colon when loops are present only increases the pain and is usually ineffective in advancing the scope through the colon. Pain increases the need for sedative and analgesic medications, which can expose the patient to more cardiorespiratory risk.

During a colonoscopy, straightness of the device is necessary, or at least highly desirable, for the advancement of the device through the colon of the patient. During the procedure, a colonoscope, a long, flexible, tubular instrument, transmits an image of the lining of the colon so the doctor can examine it for any abnormalities. The colonoscope is inserted through the rectum and advanced to the other end of the large intestine. As the scope bends, the doctor can move it around the curves of the colon to look for any abnormalities. The scope is further configured to blow air into the colon, which expands the colon and makes it easier for a doctor to look for any abnormalities or changes (e.g., swollen, irritated tissues, polyps or cancer). Once the device is inserted and withdrawn from the patient, the colon is generally insufflated either with carbon dioxide or air to improve visualization during the colonoscopy process. However, in many patients, weak anal sphincter tones causes air loss, thus requiring manual pressure, prolonging the procedure and thus increasing risk for the patient.

Another key disadvantage is that in conventional colonoscopy procedures air is introduced to distend the colon for advancement and navigation of the colonoscope and for proper visualization of possible growths. During colonoscopy, the air introduced can produce a sense of bloating and discomfort for the patient. Ultimately, air is removed by the distal end or tip of the colonoscope (which is generally the location of visual or other sensing equipment) or is passed out the rectum. Most air escapes during the procedure through the rectum but due to partial obstruction by the scope or colon anatomy, air is often trapped at various points along the colon. Currently patients in discomfort due to trapped air can either remain that way for the remainder of the procedure, attempt to pass the gas on their own, or receive more medication. Additional medication for sedation increases the risk of respiratory depression of the patient and lengthens the recovery time for the patient. Therefore, a need exists for an improved colonoscope that reduces the amount of excess air which contributes to discomfort.

Finally, currently available designs and configurations of colonoscopy devices do not address the problem related to any weakness of the sphincter muscles. The novel apparatus and method disclosed herein overcomes many of the challenges of the conventional devices and methods of colonoscopy. The present disclosure is directed to an apparatus and method comprising an improved air retention device or patch for colonoscopy. More particularly, the present disclosure is directed to an improved apparatus and method while overcoming prior art shortcomings and disadvantages related to the potential cross-contamination.

### BRIEF SUMMARY OF THE INVENTION

For the purposes of promoting and understanding the principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed and illustrated herein are contemplated as would normally occur to one of ordinary skill in the art to which this disclosure relates. Embodiments disclosed in the present disclosure provide an apparatus and a method comprising an improved air retention device or patch shaped and configured to be applied during a colonoscopy procedure.

In an aspect of the present disclosure, an improved air retention device or patch comprising a first end and a second end located at a distal end of the first end is provided. The first end and the second end further include a means of attachment for attaching a back surface of the second end to a front surface of the first end. The patch further comprises of a tightening strap formed as an integral portion of the patch. The second end of the air retention device is shaped and configured to be pulled and stretched away from the first end such that the device can be wrapped around a central axis aligned with the center of the anus of a person and attached to the first end for a colonoscopy procedure. The device can be made of a silicone or other equivalent material that can be shaped and configured to allow flexibility during the colonoscopy procedure. Further, the device disclosed herein can be applied at the beginning and/or during the colonoscopy procedure and comprises means of attachment comprises of adhesive, Velcro or other equivalent attachment means. By way of example and not of limitation, the device can be placed on the gluteus of a person and tightened around a colonoscope such that there will be no need to remove the colonoscope during a colonoscopy procedure. The device can be manufactured for both a smaller size and a larger size patient.

In another aspect of the present disclosure, a method of using a patch during a colonoscopy comprises the steps of (1) placing a first end of the patch on the gluteus of a person; (2) pulling a second end located at a distal end of the first end away from the first end; (3) providing means of attachment to attach a front surface of the first end to a back surface of the second end; (4) wrapping the patch around a colonoscope such that a central axis of the wrapped patch aligns with the center of the anus of a person; and (5) attaching the back surface of the second end to the front surface of the first end for a colonoscopy procedure. The method disclosed herein further provides a reduction of water usage and air/carbon dioxide during the colonoscopy procedure.

The term “scope” is used to refer to an endoscope, colonoscope, sigmoidoscope, proctoscope, gastroscope or other types of medical endoscopes that are or can be utilized to perform a colonoscopy procedure. As known to those skilled in the art, a typical endoscope generally comprises a connecting tube, a handle and an insertion tube (the part inserted into the patient). As disclosed herein, the term “scope” generally refers to the insertion tube portion of the endoscope or colonoscope. In referring to the opposite ends of the scope, the “proximal end” refers to that part of the scope or guide which is closest to the operator or physician endoscopist performing the procedure, and the “distal end” refers to that part of the scope or guide farthest from the operator or physician endoscopist. Although medically the rectum is generally not considered to be part of the colon, which includes the sigmoid colon, descending colon, transverse colon and ascending colon, for purposes of the present disclosure general references herein to the term “colon” include the rectum. In addition, any terms such as terms “splint,” “splinting device,” “splinting overtube” and “overtube” are used interchangeably to refer to a generally elongated hollow tubular member that is adaptable for receiving a medical instrument, such as an endoscope, therein to facilitate movement of the scope through the colon. During colonoscopy, these splinting overtubes are all utilized to overcome the problem of looping in the sigmoid colon. These overtubes are all flexible in order to extend from the anus to the descending colon, forming a bridge to prevent the sigmoid from looping, thus facilitating scope advancement.

In an aspect of the present disclosure, an air retention device for colonoscopy is provided. The device primarily comprises a material shaped and configured to be applied during the colonoscopy, placed on the gluteus and around the colonoscope so that there would be no need to remove the colonoscope.

This summary is provided to introduce a selection of concepts in a simplified form that are further described in the detailed description of the disclosure. This summary is not intended to identify key or essential inventive concepts of the claimed subject matter, nor is it intended for determining the scope of the claimed subject matter. The references made above in detail to the embodiments of the disclosure are provided by way of explanation of the disclosure, not in limitation of the disclosure. It will be apparent to those skilled in the art that various modifications and variations can be made in the present disclosure without departing from the scope or spirit of the disclosure.

Features illustrated or described as part of one embodiment can be used on another embodiment to yield a still further embodiment. Thus, it is intended that the present disclosure cover such modifications and variations as come within the scope of the appended claims and their equivalents. Other objects, features and aspects of the present disclosure are disclosed in the following detailed description. It is to be understood by one of ordinary skill in the art that the present discussion is a description of exemplary embodiments only and is not intended as limiting the broader aspects of the present disclosure, which broader aspects are embodied in the exemplary constructions.

### BRIEF DESCRIPTION OF THE DRAWINGS

The disclosure will be more readily understood in view of the following description when accompanied by the below figures. The accompanying figures incorporated in and forming a part of the specification illustrate several aspects of the present invention and, together with the description, serve to explain the principles of the invention.

FIG. 1 illustrates an embodiment of the colonoscopy procedure according to one aspect of the present disclosure.

FIG. 2 illustrates an embodiment of the improved air retention device according to one aspect of the present disclosure.

FIGS. 3 and 4 illustrate various embodiments of the colonoscopy procedure using the improved air retention device according to one or more aspects of the present disclosure.

### DETAILED DESCRIPTION OF THE DISCLOSURE

For the purposes of promoting and understanding the principles disclosed herein, reference is now made to the preferred embodiments illustrated in the drawings, and specific language is used to describe the same. It is understood that no limitation of the scope of the disclosure is hereby intended. Such alterations and further modifications in the illustrated apparatus and such further applications of the principles disclosed and illustrated herein are contemplated as would normally occur to one of ordinary skill in the art to which this disclosure relates.

A colonoscope (or scope) generally comprises of a body portion, at least one distal port located on the body portion of said colonoscope, a distal tip portion of said colonoscope body portion, a video sensor, a colonoscope handle, a light source, computer, and at least one channel for directing fluid. The colonoscope can further comprise of a plurality of motors on the handle capable of orienting the colonoscope distal tip portion in any direction of side bending, and wherein each of the motors is located on the colonoscope handle and is capable of bidirectional movement, simultaneous movement and synchronous movement with all of the motors.

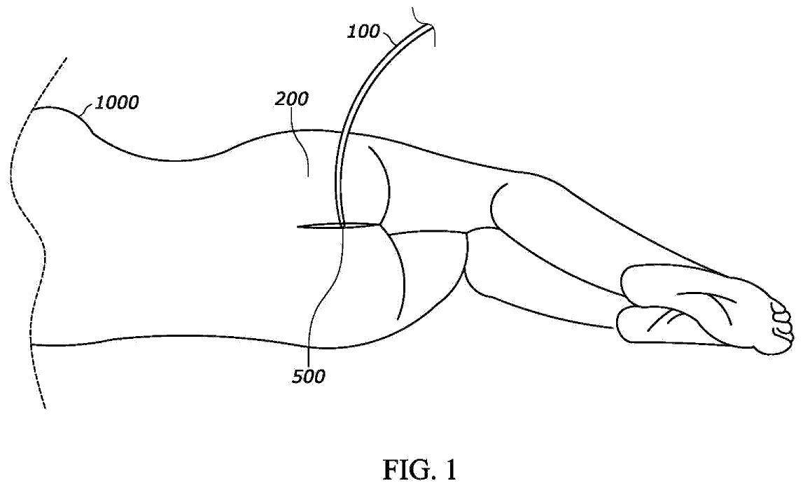

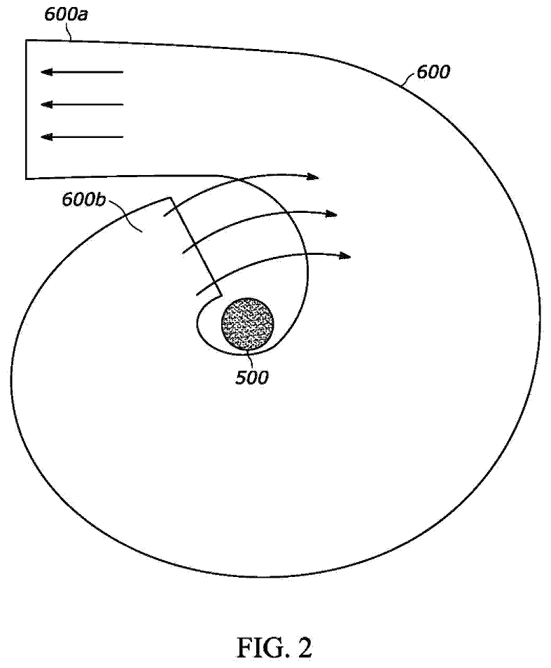

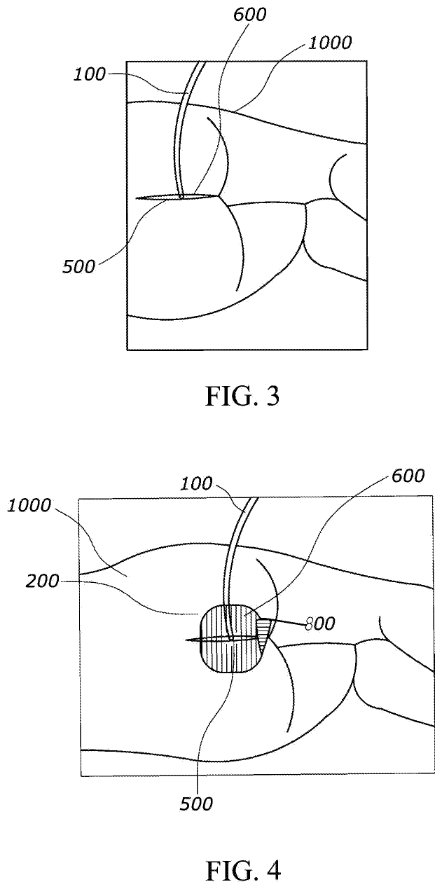

FIG. 1 illustrates an embodiment of the colonoscopy procedure according to one aspect of the present disclosure. FIG. 2 illustrates an embodiment of the improved air retention device according to one aspect of the present disclosure. FIGS. 3 and 4 illustrate various embodiments of the colonoscopy procedure using the improved air retention device according to one or more aspects of the present disclosure.

As illustrated in FIG. 1, in a standard colonoscopy procedure using a conventional colonoscope body 100 having at least one substantially non-distal port on the colonoscope body 100 being inserted into the patient body cavity 500; for example, in the anus 500 between the glutes 200 of a patient 1000. By way of example and not of limitation, in addition the colonoscope 100 comprises at least one insertion port, capable of inserting tools and fluid which remains outside a patient 1000 body cavity (e.g., anus 500), and at least one distal port, and a low friction metal or plastic woven or cast porous screen attached at an opening of one or more of the non-distal ports—the screen configured to be positioned, if needed, for preventing mucosa from entering while allowing fluid to travel to and from the substantially non-distal port on the colonoscope body.

In an aspect of the present disclosure, and as disclosed in FIG. 2, an improved air retention device 600 for use during the colonoscope procedure is provided. The device 600 primarily comprises of a material shaped and configured to be applied to the gluteus 200 region of a patient 1000 in order to reduce any patient 1000 discomfort associated with a colonoscopy procedure. The device 600 can be manufactured from materials such as silicone or other types of rubber (or other equivalent materials with similar material properties and characteristics) that could be applied to the gluteus region 200 (shown in FIGS. 1-4) of a patient 1000 during the colonoscopy when needed.

In another aspect of the present disclosure, and as illustrated in FIG. 2, an improved air retention device 600 comprises a first end 600a and a second end 600b located at a distal end of the first end 600a. The first end 600a and the second end 600b further include a means of attachment for attaching a back surface of the second end 600b to a front surface of the first end 600a. The second end 600b of the air retention device 600 is shaped and configured to be pulled and stretched away from the first end 600a such that the device 600 can be wrapped around a central axis aligned with the center of the anus 500 of a person (patient 1000) and attached to the first end 600a (via tightening as shown by the arrows at the first end 600a and second end 600b) during a colonoscopy procedure.

In certain alternative embodiments, and as illustrated in FIGS. 2 through 4, the improved air retention device 600 comprises of a patch that can be applied to the gluteus 200 region of a patient 1000 and provides the same functionality as the air retention device. The improved air retention device or the patch (600) can be placed on the gluteus 200 of a person and tightened around a colonoscope such that there will be no need to remove the colonoscope 100 during a colonoscopy procedure.

As further illustrated in the FIGS. 1 through 4, and in yet another aspect of the present disclosure, the improved air retention device 600 is made of a silicone or other equivalent material that can be shaped and configured to allow flexibility during the colonoscopy procedure. By way of example and not of limitation, the improved air retention device 600 can be applied at the beginning and/or during the colonoscopy procedure. Various means of attachment are provided as part of the improved device, e.g., attachment means can comprise of adhesive, Velcro or other equivalent attachment alternatives providing the same or improved functionality as an adhesive and/or Velcro.

In still yet another aspect of the present disclosure, the usage of the device can reduce the amount of water and air/carbon dioxide during the colonoscopy procedure. The improved air retention device can be manufactured for both a smaller size and a larger size patient.

As shown in FIG. 2, the improved air retention device 600 disclosed herein can be applied as the medical staff (the endoscopist/doctor) finds it necessary during a colonoscopy procedure. In certain embodiments, the air retention device 600 comprises of a first end and a second end located at a distal end of the first end. During colonoscopy, the first end 600a of the air retention device is put on the surface of a person's buttocks—placed on the gluteus 200 of a person—such that the surface of the first end 600a is flushed with the skin of the gluteus 200 of a person. Next, the second end 600b of the air retention device 600 is pulled away from the first end 600a and tightened in an anticlockwise direction while the first end is pulled in a clockwise direction—as shown in FIG. 2—the device 600 configured to wrap around a central axis aligned with the center of the anus 500 of the person/patient 1000 undergoing the colonoscopy procedure.

As illustrated in FIGS. 1-4, as the colonoscope 100 is inserted through the rectum of a person and advanced to the other end of the intestine, the scope bends, so that the doctor can move it around the curves of the colon and inspect for any abnormalities within the person's colon. During the procedure, the scope can also blow air into the colon, which will expand the colon and help the doctor inspect and note any abnormalities more clearly. The novel and unique configuration of the improved air retention device and method, disclosed herein, reduces patient discomfort by reducing the amount of water and air/carbon dioxide during the colonoscopy procedure.

In an aspect of the present disclosure, a method of using the air retention device 600 during colonoscopy comprises the steps of (1) placing a first end 600a of the patch on the gluteus of a person; (2) pulling a second end 600b located at a distal end of the first end 600a away from the first end 600a; (3) providing means of attachment to attach a front surface of the first end 600a to a back surface of the second end 600b; (4) wrapping the patch around a colonoscope 100 such that a central axis of the wrapped patch aligns with the center of the anus 500 of a person 1000; and (5) attaching the back surface of the second end 600b to the front surface of the first end 600a for a colonoscopy procedure. In certain alternative embodiments, the improved air retention device 600 comprises of a patch that can be applied to the gluteus region 200 of a patient and provide the same functionality as the air retention device. It is to be noted, by way of example and not of limitation, that the improved air retention device or patch 600 can be placed on the gluteus 200 of a person and tightened around a colonoscope 100 such that there would be no need to remove the colonoscope 100 during a colonoscopy procedure.

In an aspect of the present disclosure, and as shown in FIG. 4, an air retention device 600 comprising a tightening strap 800 formed as an integral portion of the second end 600b is provided. The tightening strap 800 comprises of a front and a back surface wherein the front surface comprises of fastening means such as adhesive, Velcro or other types of means of adhering to the first end of the air retention device. By way of example and not of limitation, once the air retention device 600 is flush with the surface of a person's buttocks 200— the surface of the first end is flush with the skin of the person's buttocks—the second end 600b of the air retention device 600 is pulled away from the first end around a central axis aligned with the center of the anus of the person undergoing the colonoscopy. The method of using the air retention device with the integrally formed tightening strap 800 during a colonoscopy comprises the steps of (1) placing a first end 600a of the patch 600 on the gluteus of a person; (2) pulling a second end 600b having the integrated tightening strap 800 located at a distal end of the first end 600a away from the first end 600a; (3) providing means of attachment to attach a front surface of the first end 600a to a back surface of the integral tightening strap 800 of the second end 600b; (4) wrapping the patch 600 around a colonoscope 100 such that a central axis of the wrapped patch 600 aligns with the center of the anus 500 of a person/patient 1000; and (5) attaching the back surface of the integral tightening strap 800 of the second end 600b to the front surface of the first end 600a for a colonoscopy procedure.

The preceding description, therefore, is not meant to limit the scope of the disclosure but to provide sufficient disclosure to allow one of ordinary skill in the art to practice the disclosure without undue burden. It is further understood that the scope of the present disclosure fully encompasses other embodiments that may become obvious to those skilled in the art, and that the scope of the present disclosure is accordingly limited by nothing other than the appended claims.

The use of the terms “a”, “an,” “the” and similar referents in the context of describing the invention (especially in the context of the following claims) is to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

The methods described and disclosed herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

It is understood that the preceding is merely a detailed description of some examples and embodiments of the present disclosure, and that numerous changes to the disclosed embodiments may be made in accordance with the disclosure made herein without departing from the spirit or scope of the disclosure. The preceding description, therefore, is not meant to limit the scope of the disclosure, but to provide sufficient disclosure to allow one of ordinary skill in the art to practice the disclosure without undue burden.

It is further understood that the scope of the present disclosure fully encompasses other embodiments that may become obvious to those skilled in the art. Features illustrated or described as part of one embodiment can be used in another embodiment to yield a still further embodiment.

Thus, it is intended that the present disclosure cover such modifications and variations as come within the scope of the appended claims and their equivalents. It is to be understood by one of ordinary skill in the art that the present discussion is a description of exemplary embodiments only, and is not intended as limiting the broader aspects of the present disclosure, which broader aspects are embodied in the exemplary constructions.

The terms “invention,” “the invention,” “this invention,” “the present invention,” “disclosure,” “the disclosure” and “the present disclosure” used in this patent are intended to refer broadly to all of the subject matter of this patent and the patent claims below. Statements containing these terms should be understood not to limit the subject matter described herein or to limit the meaning or scope of the patent claims below.

Embodiments of the invention covered by this patent are defined by the claims below. This specification is a high-level overview of various aspects of the invention and introduces some of the concepts that are further described in the detailed description. This specification is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim. Although the present disclosure and its advantages have been described in detail, it should be understood that various changes, substitutions and alterations can be made herein without departing from the spirit and scope of the disclosure as defined in the appended claims.

1. An improved air retention device comprising
a first end;
a second end located at a distal end of the first end;
wherein the first end and the second end further include means of attachment for attaching a back surface of the second end to a front surface of the first end; and
wherein the second end of the air retention device is shaped and configured to be pulled and stretched away from the first end such that the device can be wrapped around a central axis aligned with the center of the anus of a person and attached to the first end for a colonoscopy procedure.

2. The improved air retention device of claim 1, where the device is made of a silicone or other equivalent material that can be shaped and configured to allow flexibility during the colonoscopy procedure.

3. The improved air retention device of claim 1, wherein the device can be applied at the beginning and/or during the colonoscopy procedure.

4. The improved air retention device of claim 1, wherein the means of attachment comprises of adhesive, Velcro or other equivalent attachment means.

5. The improved air retention device of claim 1, wherein the device can be placed on the gluteus of a person and tightened around a colonoscope such that there will be no need to remove the colonoscope during a colonoscopy procedure.

6. The improved air retention device of claim 1, wherein the usage of the device can reduce the amount of water and air/carbon dioxide during the colonoscopy procedure.

7. The improved air retention device of claim 1, wherein the device can be manufactured for both a smaller size and a larger size patient.

8. A patch comprising
a first end;
a second end located at a distal end of the first end;
wherein the first end and the second end further include means of attachment for attaching a back surface of the second end to a front surface of the first end; and
wherein the second end of the patch is shaped and configured to be pulled and stretched away from the first end such that the patch can be wrapped around a central axis aligned with the center of the anus of a person and attached to the first end for a colonoscopy procedure.

9. The patch of claim 8, where the patch is made of a silicone or other equivalent material that can be shaped and configured to allow flexibility during the colonoscopy procedure.

10. The patch of claim 8, wherein the patch can be applied at the beginning and/or during the colonoscopy procedure.

11. The patch of claim 8, wherein the means of attachment comprises of adhesive, Velcro or other equivalent attachment means.

12. The patch of claim 8, wherein the patch can be placed on the gluteus of a person and tightened around a colonoscope such that there will be no need to remove the colonoscope during a colonoscopy procedure.

13. The patch of claim 8, wherein the device can be manufactured for both a smaller size and a larger size patient.

14. A method of using a patch during colonoscopy, the method comprising the steps of:
placing a first end of the patch on the gluteus of a person;
pulling a second end located at a distal end of the first end away from the first end;
providing means of attachment to attach a front surface of the first end to a back surface of the second end;
wrapping the patch around a colonoscope such that a central axis of the wrapped patch aligns with the center of the anus of a person; and
attaching the back surface of the second end to the front surface of the first end for a colonoscopy procedure.

15. The method of claim 14, where the device is made of a silicone or other rubber material that can be shaped and configured to allow flexibility during the colonoscopy procedure.

16. The method of claim 14, wherein the device can be applied at the beginning and/or during the colonoscopy procedure.

17. The method of claim 14, wherein the means of attachment comprises of adhesive, Velcro or other equivalent attachment means.

18. The method of claim 14, wherein the patch can be placed on the gluteus of a person and tightened around a colonoscope such that there would be no need to remove the colonoscope during a colonoscopy procedure.

19. The method of claim 14, wherein the patch can be manufactured for both a smaller size and a larger size patient.

20. An improved method of performing colonoscopy, the method comprising the steps of:
placing a first end of the patch on the gluteus of a person;
pulling a second end located at a distal end of the first end away from the first end;
providing means of attachment to attach a front surface of the first end to a back surface of the second end of the patch;
wrapping the patch around a colonoscope inserted into the anus of a patient such that a central axis of the wrapped patch is aligned with the center of the anus of a person;
attaching the second end to the first end for a colonoscopy procedure; and
providing a reduction of water usage and air/carbon dioxide during the colonoscopy procedure.
